(19) United States
(12) Patent Application Publication
(10) Pub. No.: US 2024/0071582 A1
(43) Pub. Date: Feb. 29, 2024

(54) LifeStory Connection

(71) Applicant: Thomas Louis Alaimo, Prospect, KY (US)

(72) Inventor: Thomas Louis Alaimo, Prospect, KY (US)

(21) Appl. No.: 18/330,309

(22) Filed: Jun. 6, 2023

Related U.S. Application Data
(60) Provisional application No. 63/366,030, filed on Jun. 8, 2022.

(51) Int. Cl. G16H 10/60; G16H 15/00; G16H 20/70

(52) U.S. Cl. CPC G16H 10/60; G16H 15/00; G16H 20/70

(57) ABSTRACT

The new type of database structure developed for the LifeStory Connection™ application was built using PHP (PHP Hypertext Preprocessor), Linux, the open-source operating system and MySQL, the relational database management system. Several processes contribute to this design making this application a new offer to the senior living industry, including dementia care, and can be applied to several other industries including special needs, mental health, etc. The build includes both physical and logical models of designs. The unique and efficient processes that the LifeStory Connection™ application combines to provide include the capturing of data remotely/virtually, the organization of the data, specific to dementia care, the storing and analyzing of the data, and the conveying of both the metrics (to facilities/communities) and resident data to staff/care partners in dementia care. The mobile app development platform used for the LifeStory Connection™ application to be conveyed to staff/care partners on tablets/devices is Flutter.

### CROSS-REFERENCE TO RELATED APPLICATION

This non-provisional patent application is a follow-up to Provisional Patent No. 63/366,030 filed Jun. 8, 1922 at 2:11:25 pm EST.

### STATEMENT OF FEDERALLY SPONSORED RESEARCH OR DEVELOPMENT

Not Applicable

### NAMES OF PARTIES TO A JOINT RESEARCH AGREEMENT

Not Applicable

### REFERENCE TO A “SEQUENCE LISTING” TABLE, OR COMPUTER PROGRAM LISTING APPENDIX SUBMITTED

The LifeStory Connection™ application was built using the following tools:
a. PHP (PHP Hypertext Preprocessor)—Server-side scripting language
b. Linux—The open-source operating system
c. MySQL—The relational database management system
d. Flutter—The mobile app development platform

### BACKGROUND ON THE INVENTION

I have been serving the senior living industry since 2003 with a focus in Dementia Care (also referred to as Memory Care), Program Development, Environmental Safety & Security, Compliance, and Disaster Preparedness. Eleven of the years I served the senior living industry I led Memory Care Operations for two large providers and Memory Care became my passion.

You have likely heard of Alzheimer's Disease. Alzheimer's is the most common cause of dementia, a general term for memory loss and other cognitive abilities serious enough to interfere with daily life. One of the most common and effective treatments for Alzheimer's and other dementia is when those caring for a person utilize a person-centered approach or person-centered care. Person-centered care involves tailoring a person's care to their interests, abilities, history, and personality.

Benefits of utilizing Person-centered care are:
Person-Centered care helps to ensure people with dementia can take part in the things they enjoy.
It can be an effective way of preventing and managing behavioral and psychological symptoms of dementia.

The key points in providing Person-centered care are:
Treating the person with dignity and respect
Understanding their history, lifestyle, culture, and preferences, including their likes, dislikes, hobbies, and interests
Looking at situations from the point of view of the person with dementia
Providing opportunities for the person to have conversations and relationships with other people
Ensuring the person has the chance to try new things or take part in activities they enjoy.

Today, most senior living memory care providers pride themselves on offering a person-centered approach to dementia care. In order to do that, they need to capture each person's/residents' life story and preferences prior to them moving into their facility/community. Residents/persons with dementia typically can't speak their needs and are not always able to provide their life story information or preferences when asked. However, by knowing this information it can help foster communication by introducing discussion about their occupation, hobbies, interests, etc.

Sadly, paper is the primary means to capture all of this information and on average, the questionnaire is 4 to 6 pages long. With all that families of loved ones with dementia are going through when they ultimately decide to move them into dementia care, they are burdened with having to handwrite their loved one's life story and preferences. Many times, handwriting can be difficult to read.

At a facility/community, managers/directors/leaders of memory care overseeing a population of persons/residents with dementia are tasked with knowing their resident population. This way, they can create a program of activities each and every day that mirror their residents' most popular/favorite activities, music, pets, hobbies, sports teams, etc. in their facility/community. It's also important to know their population's interest in religion, veteran status, and birthdays. All of this contributes to providing a Person-centered approach in dementia care. However, by having paper copies of their residents' life story and preferences, they are burdened with manually sorting through all of the paper copies to correlate similar interests. Additionally, as mentioned, many times handwriting can be difficult to read.

Finally, at the staff/care persons level, in order to provide a Person-centered approach to dementia care, staff need to learn and commonly reference each of their residents' life story and preference information. This helps to provide normalcy, choice, and purpose in the life of the persons/residents living with dementia. By having paper copies of their residents' life story and preference, many times staff find them difficult to read and the paper copies are often misplaced. Paper copies are typically kept in binders on a desk of on a shelf.

Sometimes the life story and preference information is copied on note cards, added to posters on the wall, etc.

Care Plans:

Regulatory requirements in senior living include the need for a care plan. A care plan is an organizational tool that outlines the care and services required to meet the needs of a person/resident. Care plans primarily focus on the persons/residents personal, social, activity, and medical needs as well as detail the steps needed to meet those needs/desires.

Often times in dementia care, facilitating a care plan can be challenging for staff/care partners. By utilizing the persons/residents' life story and preferences, staff/care partners can identify approach techniques and overall strategies that are familiar to the person/resident when assisting with care, engaging them in activities, during meals, etc. These strategies can significantly contribute to improving the lives of persons/residents in dementia care. Between staff/care partners and persons/residents with dementia, these strategies can help to build trust, improve engagement, and yield better outcomes. In other words, there is often less resistance to care (refusing bathing, etc.), an increased attending of activities, and overall, things go a lot smoother throughout each day.

These strategies are shared with other staff/care partners and are commonly referred to as care plan strategies.

Here is an example of how helpful it can be to staff/care partners when they know a person's life story and preferences, and utilize the care plan strategies that are learned:

### EXAMPLE

A new employee, Diane, just completed orientation and is working in a memory care facility/community. Diane is tasked with going to Jane Doe's apartment and escorting her to the dining room for lunch. Having just started her employment, Diane doesn't know Jane very well. Therefore, Diane references Jane's life story and preference information on the documentation stored in a binder on the memory care directors desk. Jane reads through the many pages and learns that Jane taught first grade for her entire career. Diane digs further into Janes life story to see what preferences she has with dining. Fortunately, the life story and preference documentation for Jane is still in the binder and she locates the information related to dining as follows:
Jane prefers sitting near the courtyard window so she can see the bird feeder during her meal and is comfortable sitting with others.
Jane prefers her vegetables to be on a separate plate than her main course.

Diane proceeds to Janes apartment, knocks on the door, and greets Jane with “Hi Jane, I'm Diane, and I'm a new employee here. I understand you were a first-grade teacher. Wow, my favorite teacher taught first grade. It's time for lunch, let's walk down to the dining room. I found the perfect table for you that overlooks the courtyard with a great view of the bird feeder . . . ”

Jane has some form of dementia and does not recognize Diane. However, based on Diane's approach and knowing some of Jane's life story and preferences, there's a good chance Jane will be receptive to greeting and accompanying Diane to the dining room. Diane's familiarity with Jane's life story has likely put Jane at ease.

Because the life story and preference information were on paper, this took Diane a great deal of time. Fortunately, in this case, it didn't negatively affect the outcome. In the real world, staffing is a challenge and finding the time to reference life story and preference information on paper is not realistic.

Important Note: LifeStory Connection™ data includes residents' life story information and personal preferences. No health-related information is captured or maintained in the LifeStory Connection™ application, databases, or tablets. All users of the LifeStory Connection™ application are informed at each login and during data entry to avoid entering any health-related information in the application, and that the Health Insurance Portability and Accountability Act (HIPAA) does not apply.

In 2022 I recognized an opportunity to design and build an innovative software application to improve the lives of persons living with dementia, their families, and care partners. This effort is also personal. As an adult child, alongside my six siblings, we experienced dementia first-hand with our Mom's journey.

By digitizing life stories and incorporating care plan strategies as they're identified, providing Person-centered care can be accomplished in a much more efficient manner and help families, staff/care partners, and improve the lives of persons/residents with dementia.

### BRIEF SUMMARY OF THE INVENTION

By creating a new type of database structure to capture, store, analyze and organize unique fields of data, the LifeStory Connection™ application was created and provides an innovative, easy to use application that digitizes the life stories, preferences, and care plan strategies of persons with dementia to support a person-centered approach to dementia care.

### BRIEF DESCRIPTION OF VIEWS OF THE DRAWING (IF ANY)

Note that all of the information provided in this Patent submission include fictional people's names, information, and photos that were permissible to share. The data is for simulation purposes only. Below is a description of the drawing views by the specified letters/numbers of each figure:

FIG. 1.—Website home page: http://www.lifestoryconnection.com: This is a snapshot of the home page of the LifeStory Connection™ website. This site outlines the LifeStory Connection™ application benefits to the public and also provides access to the unique new type of database structure of the application to families, facilities/communities, and the admins designated.

FIG. 2.—Generate Member Access Code: This is a snapshot of the Generate Member Access Code feature. This part of the application is what a facility/community would access to generate an access code to a family of a potential new person/resident. This access code, when active, grants a family access to the unique new type of database structure to enter in their loved one's life story and preferences.

FIG. 3.—Generate Resident: This is a snapshot of the Generate Resident feature. This feature of the application is what a facility/community would use to enter in an existing person/residents' life story and preference information into the unique new type of database structure (if for example, they're launching the LifeStory Connection™ application and are entering in their existing residents with the information they already gathered on paper). The facility/community may also utilize this feature if the family prefers to write their loved one's life story and preferences on paper and not utilize the application family entry feature.

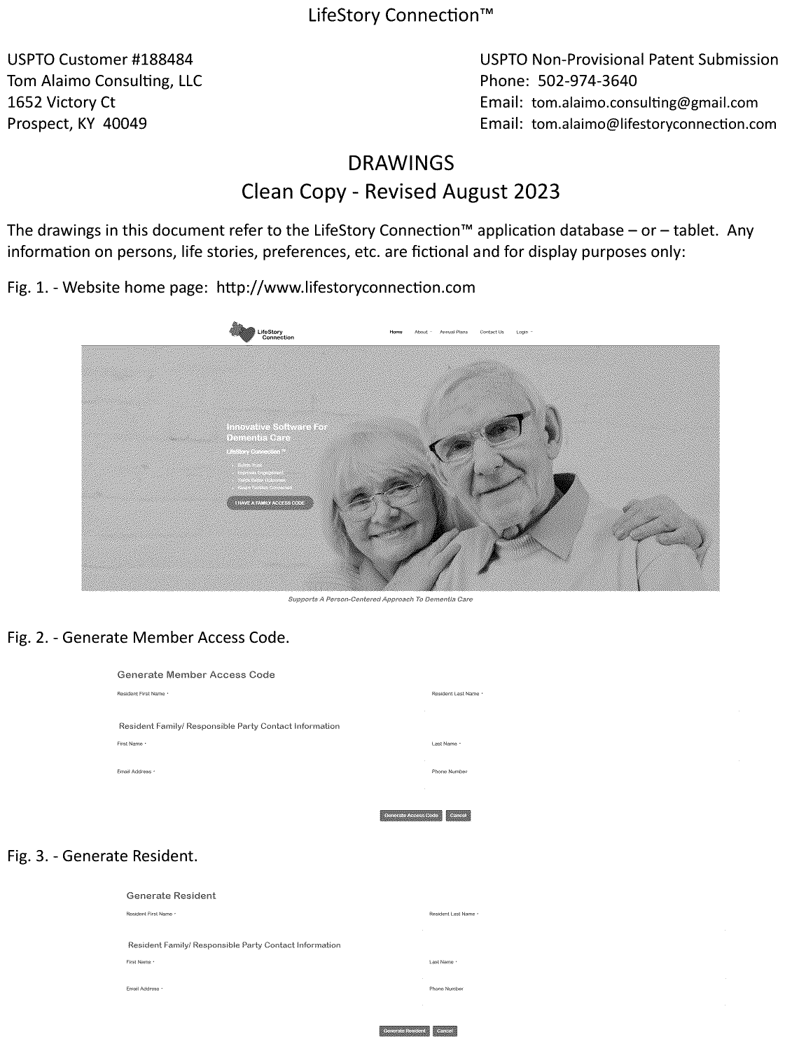

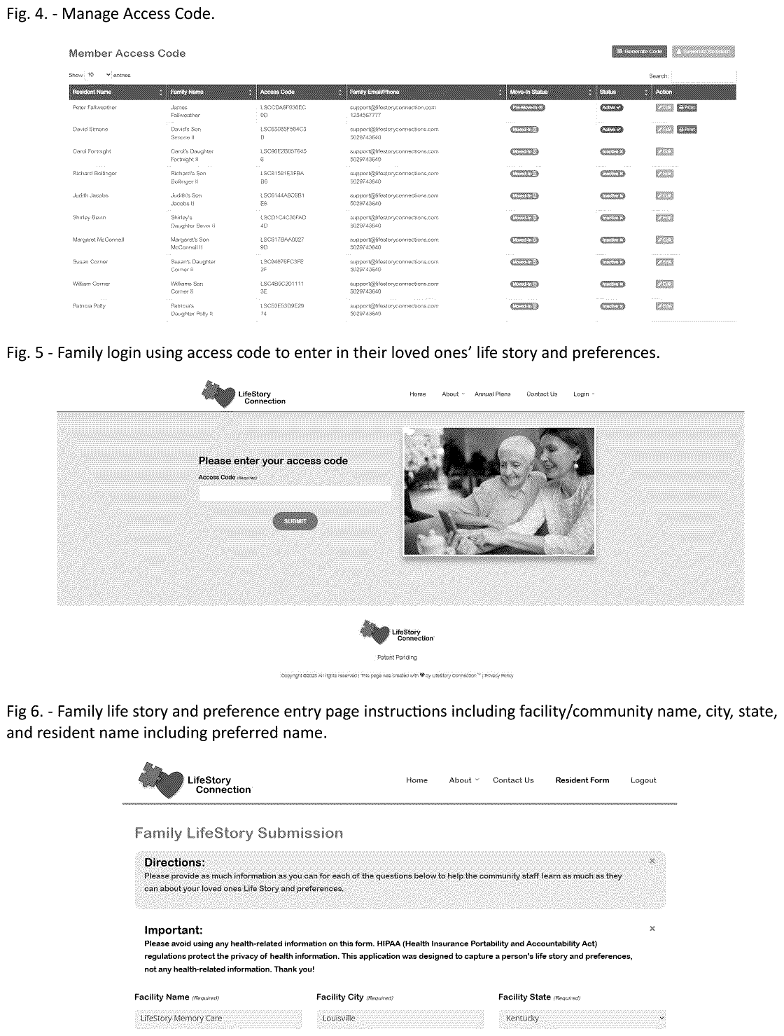

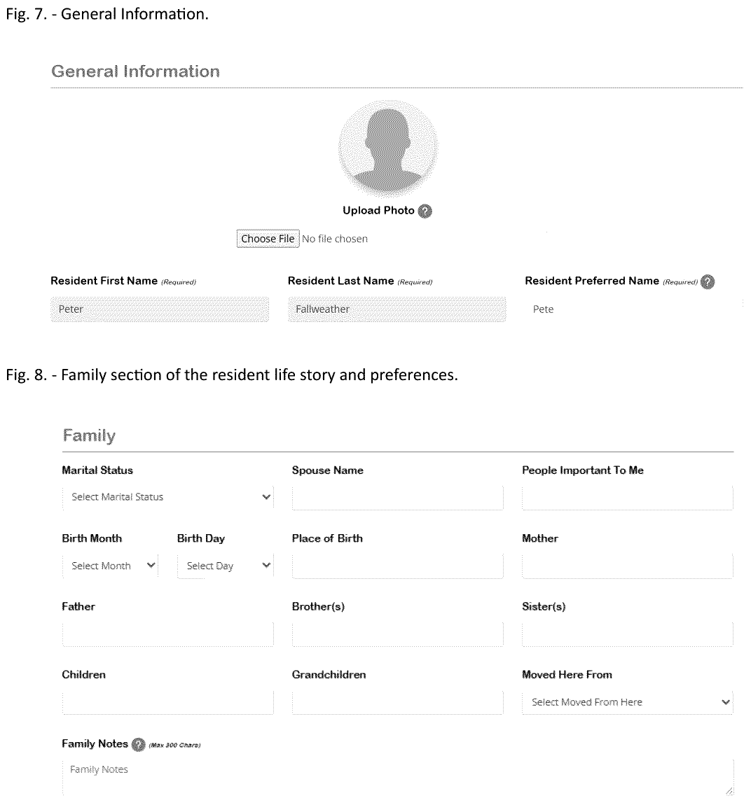

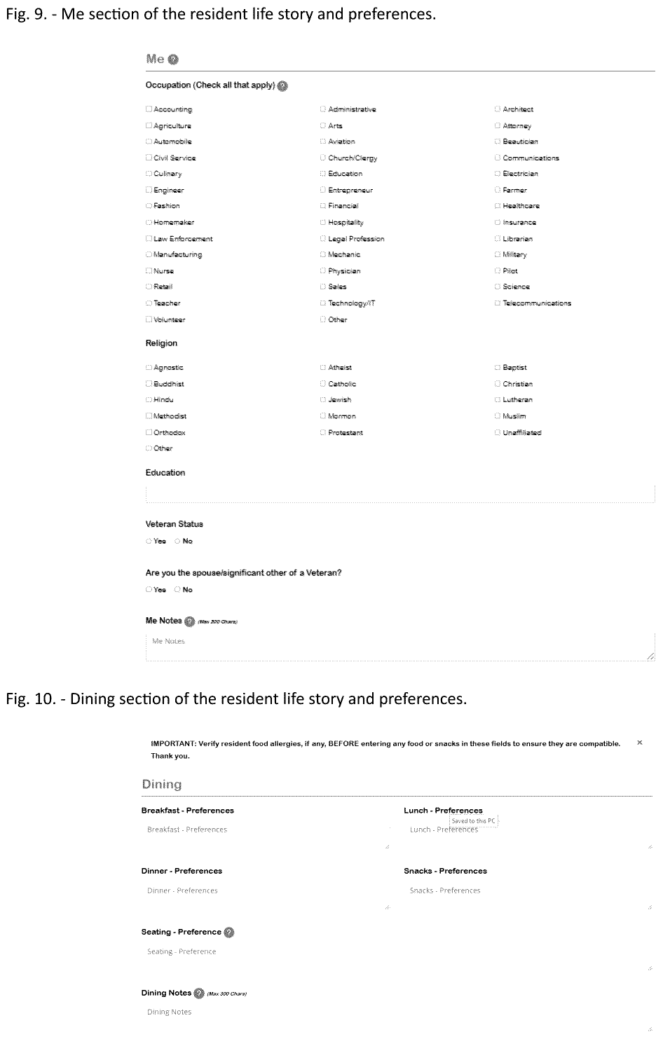

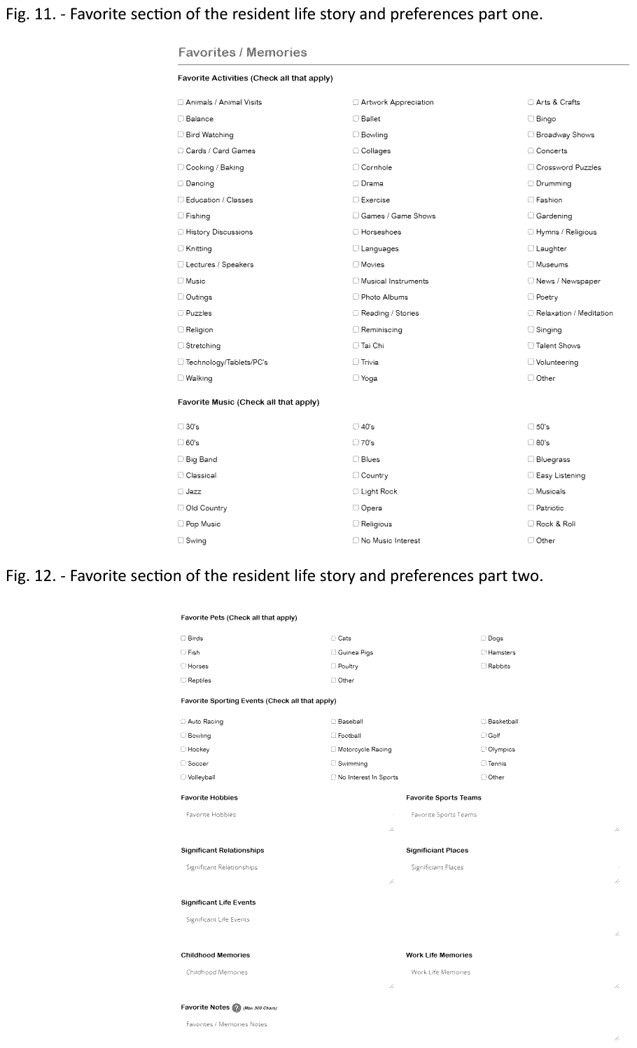

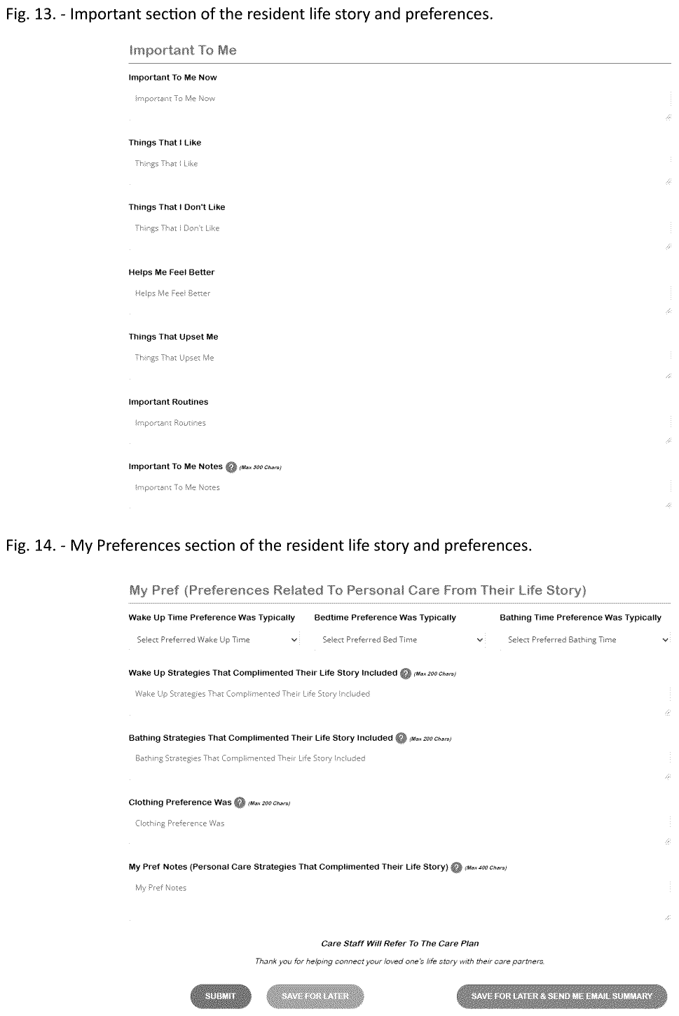

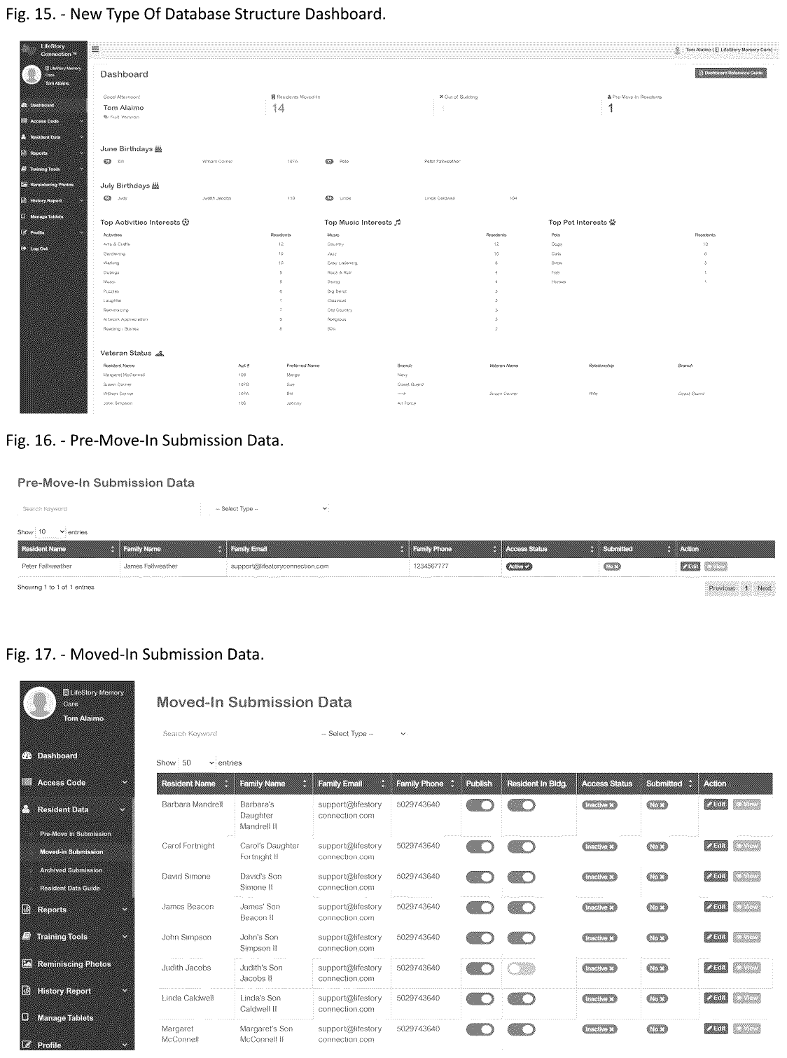

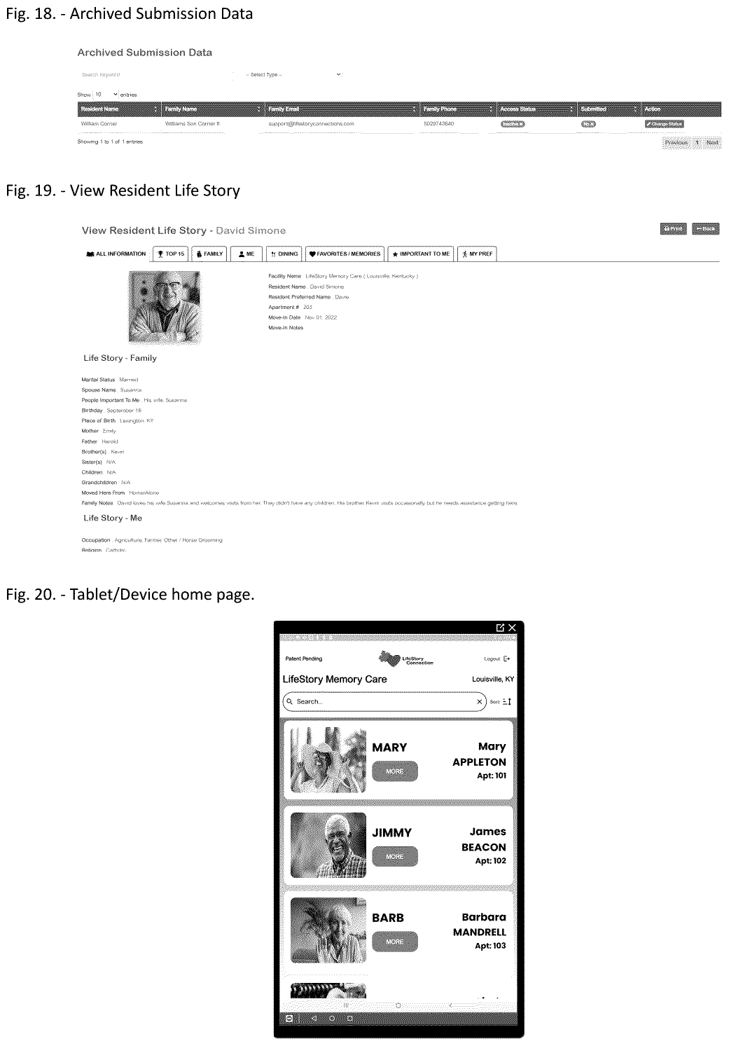

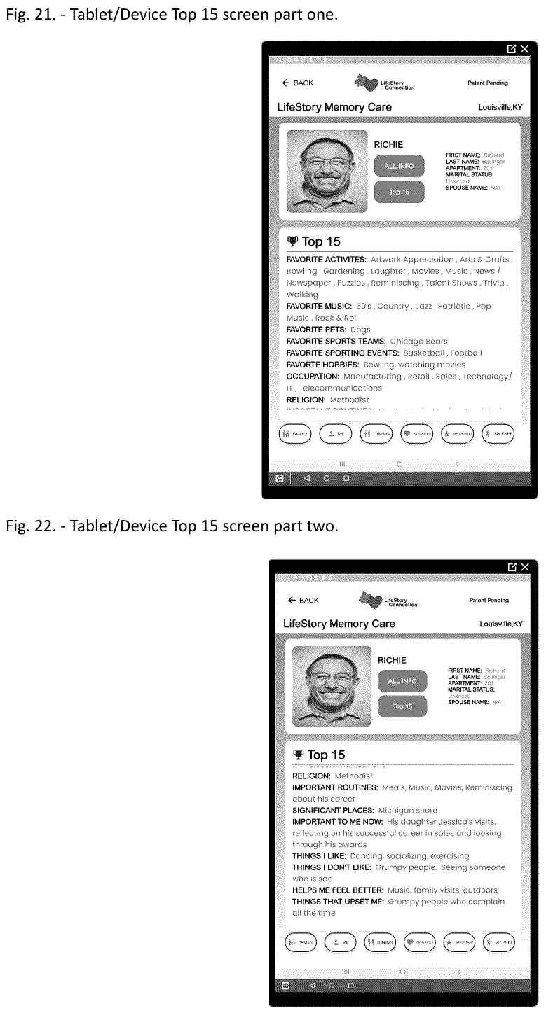

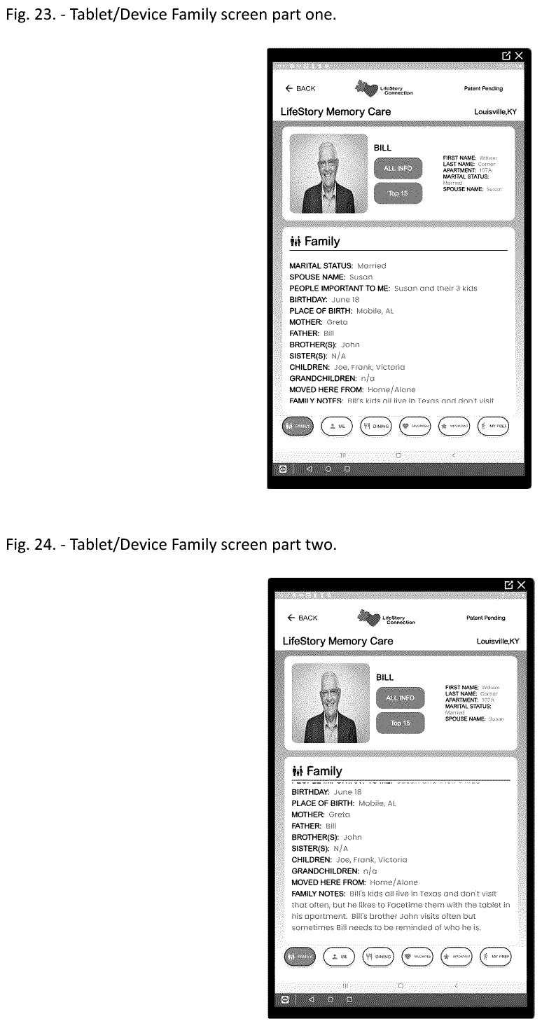

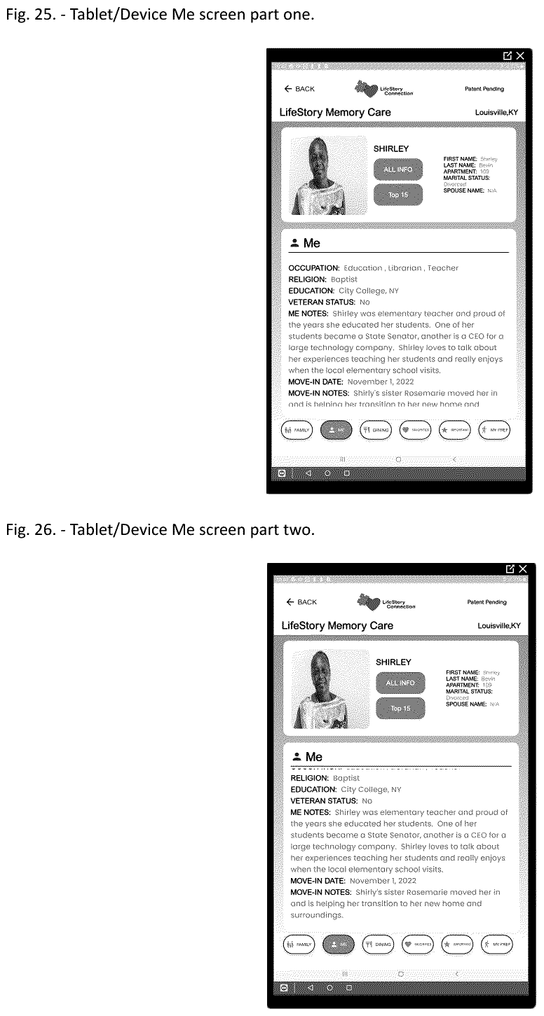

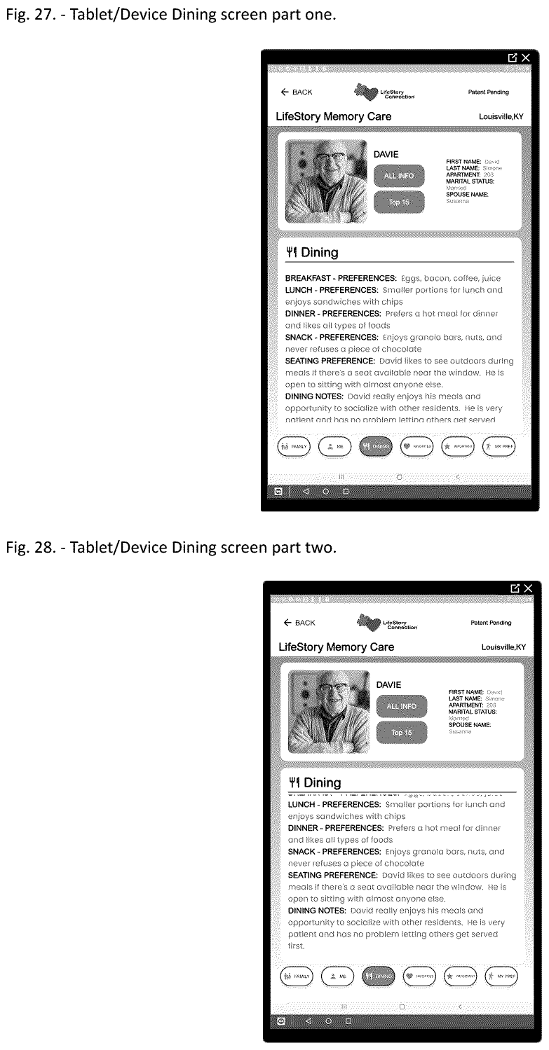

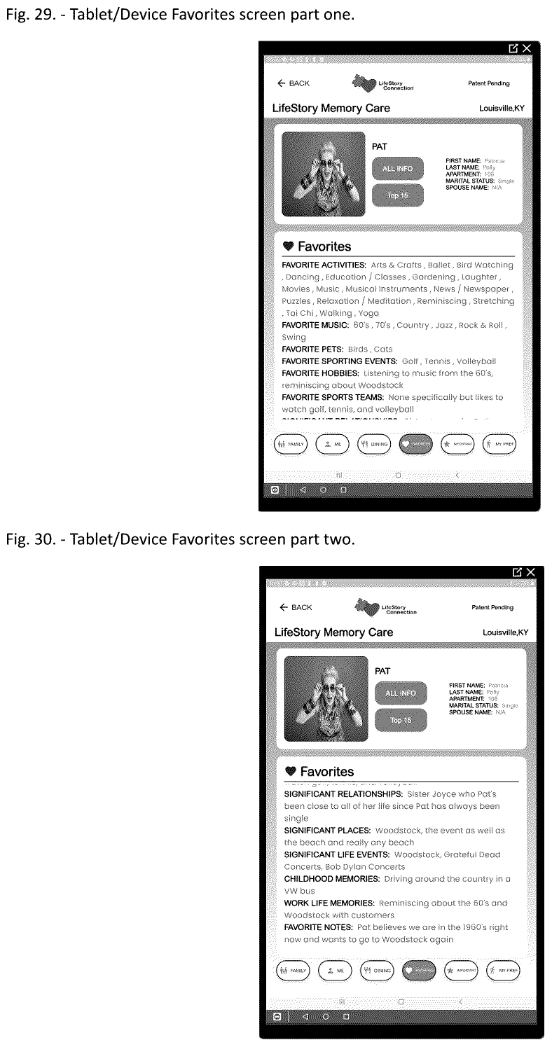

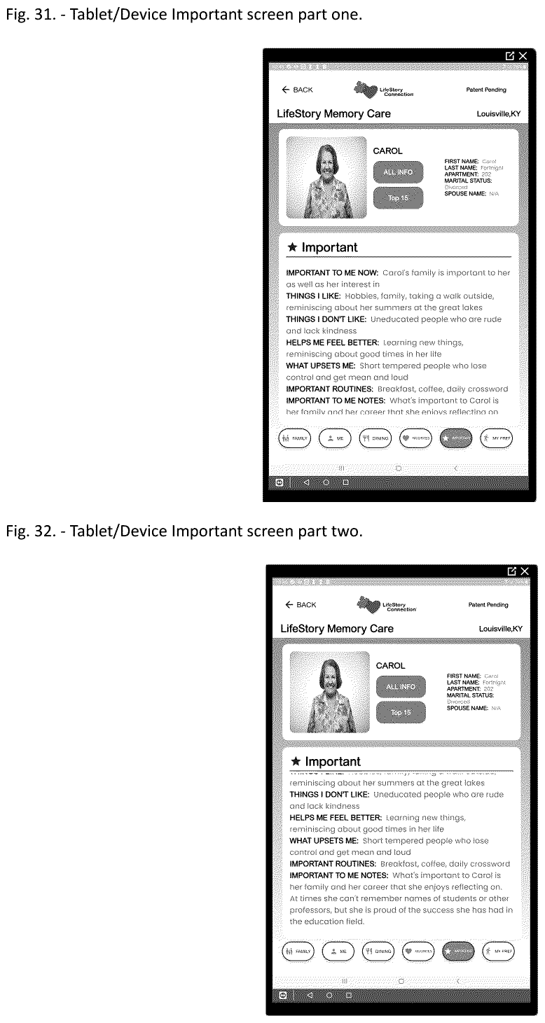

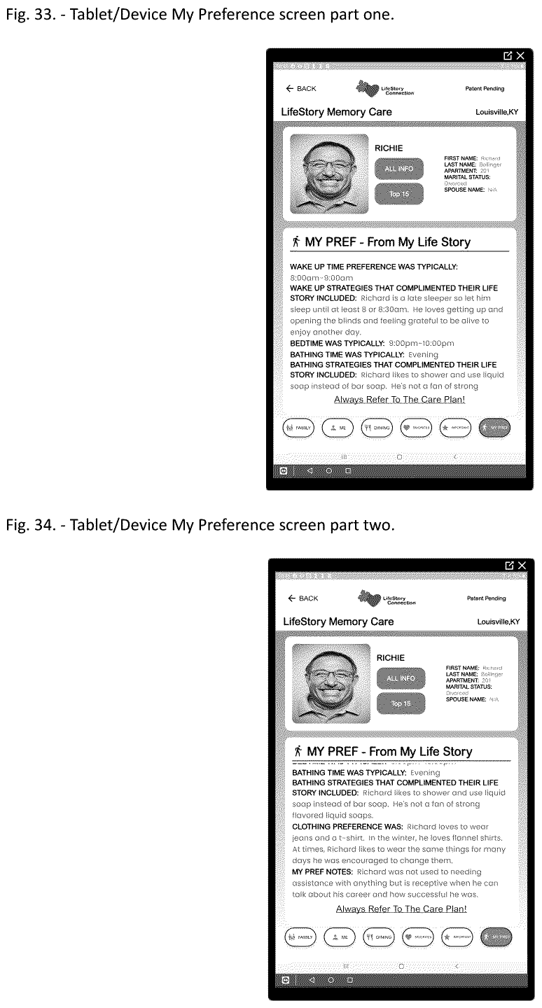

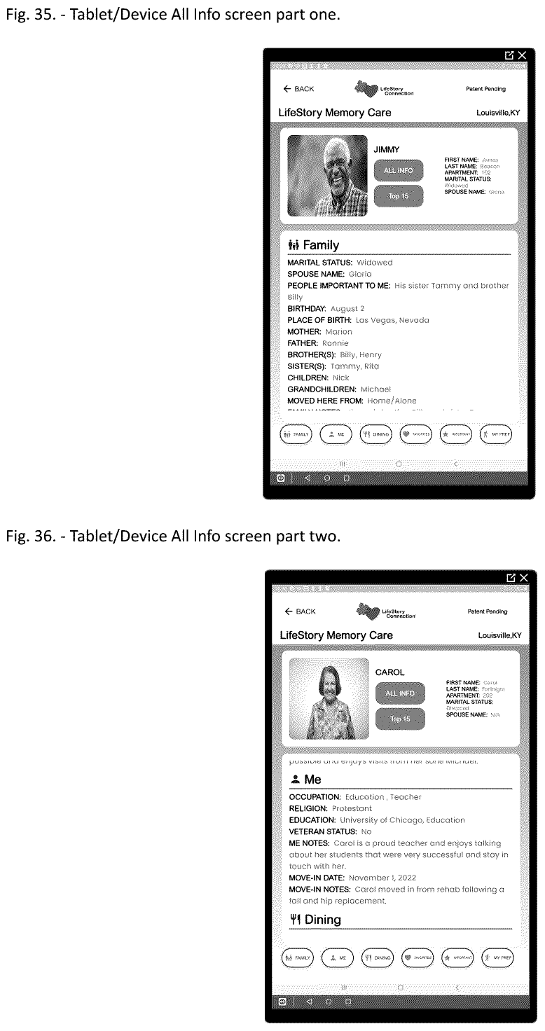

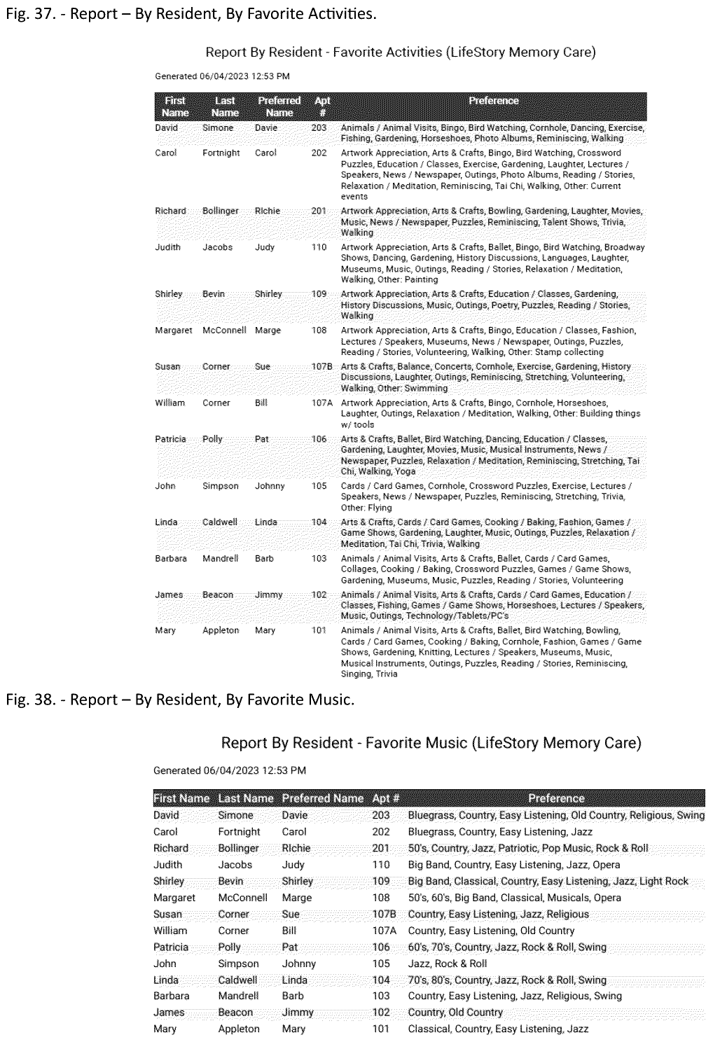

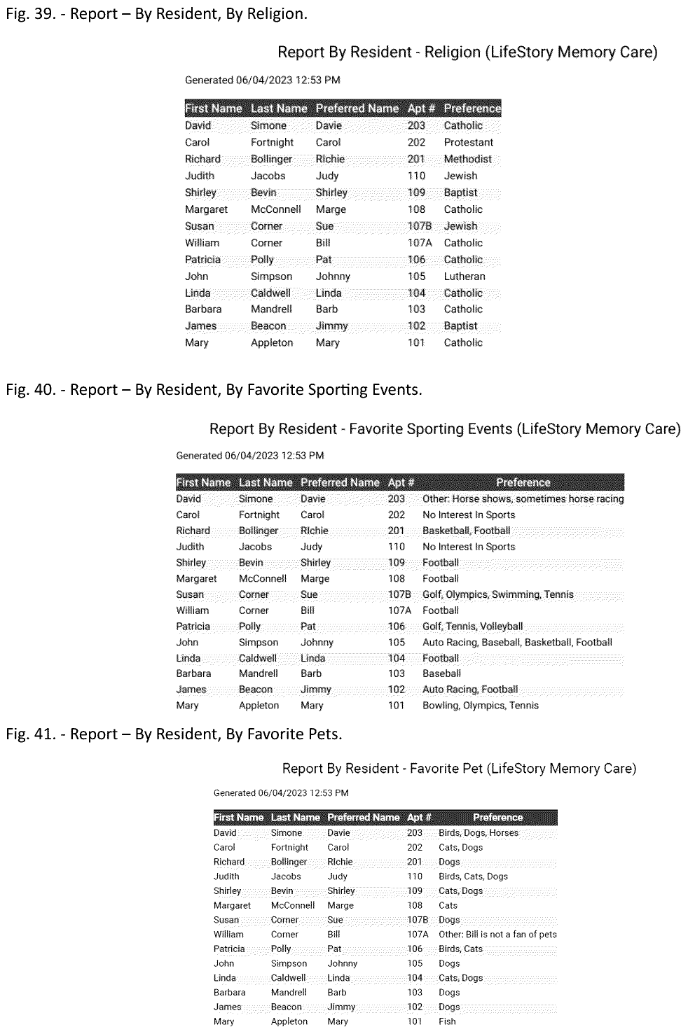

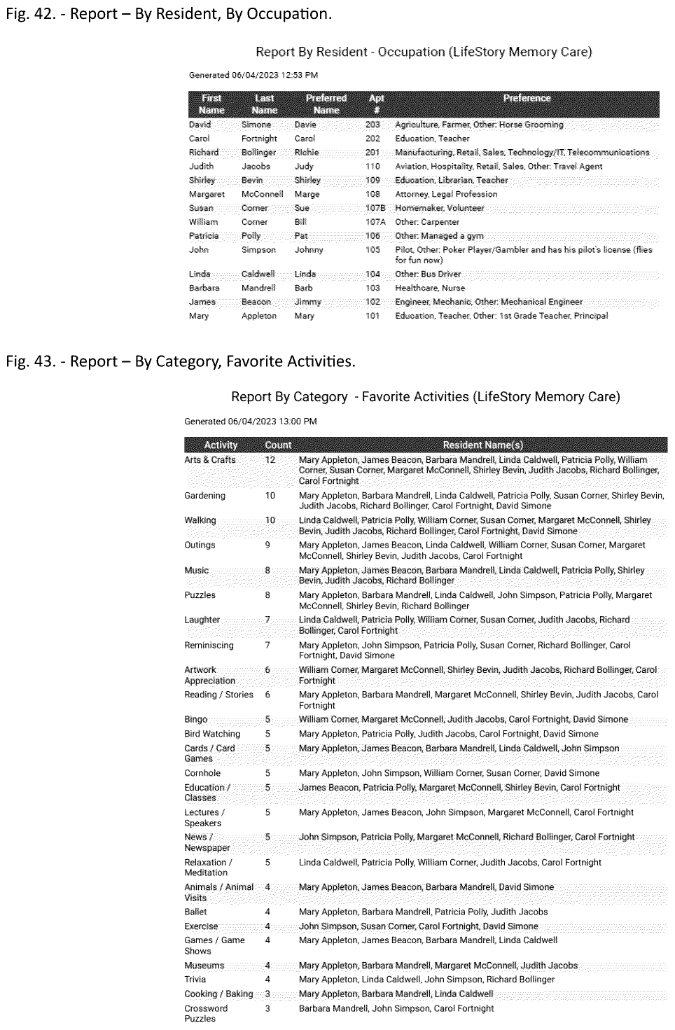

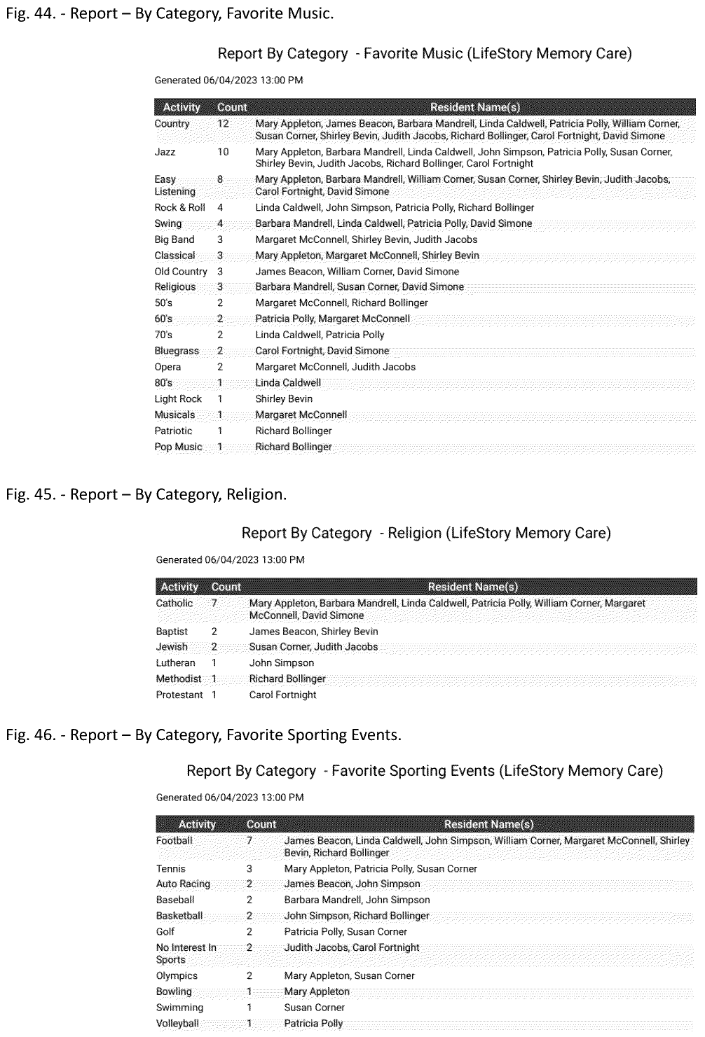

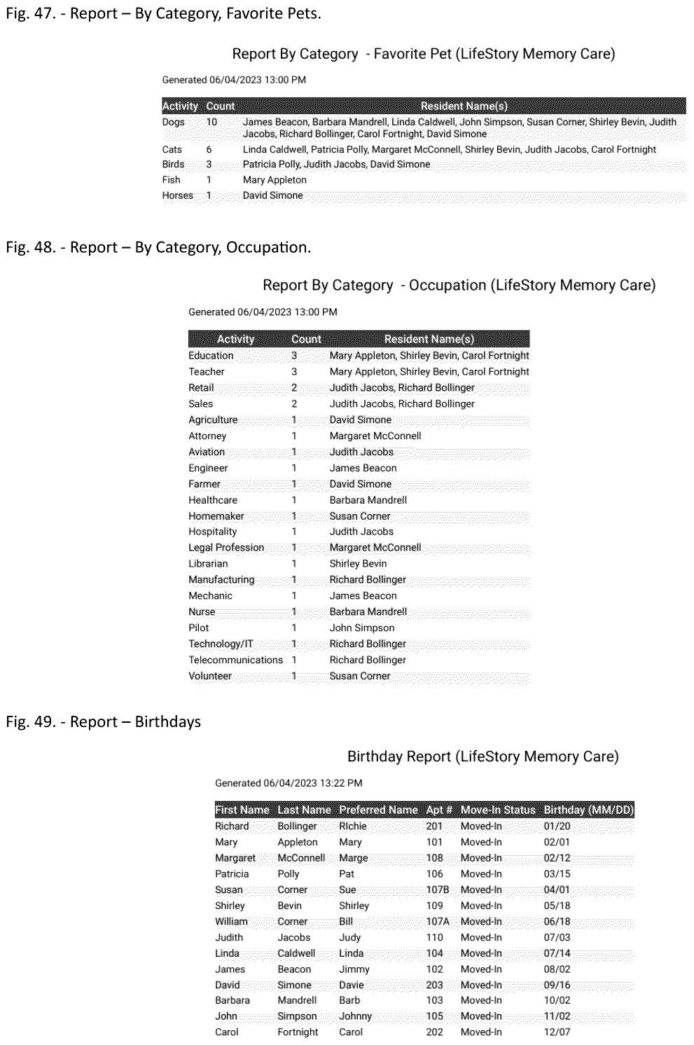

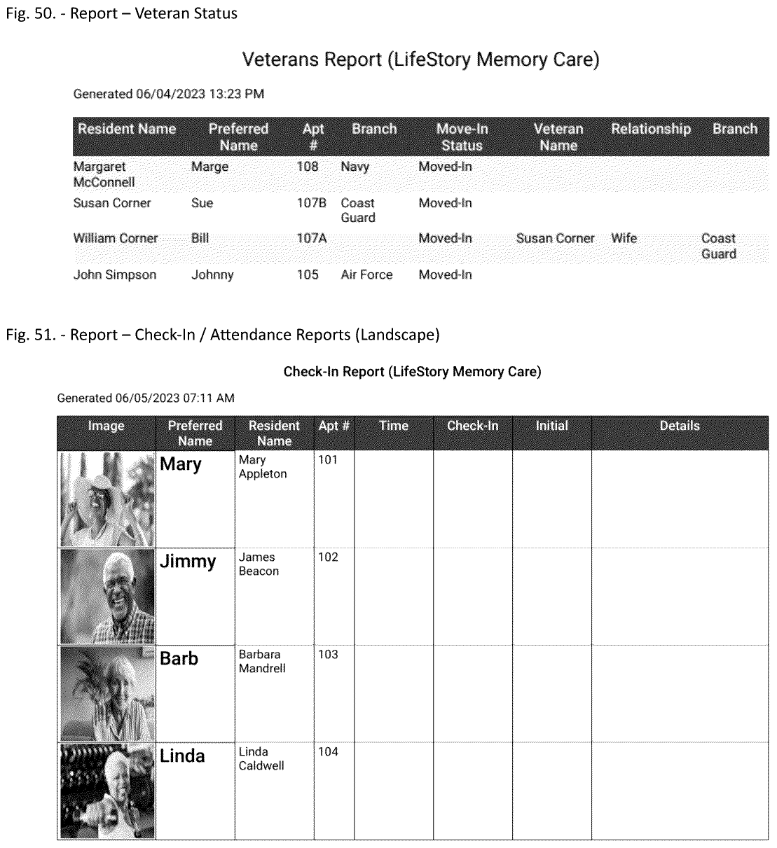

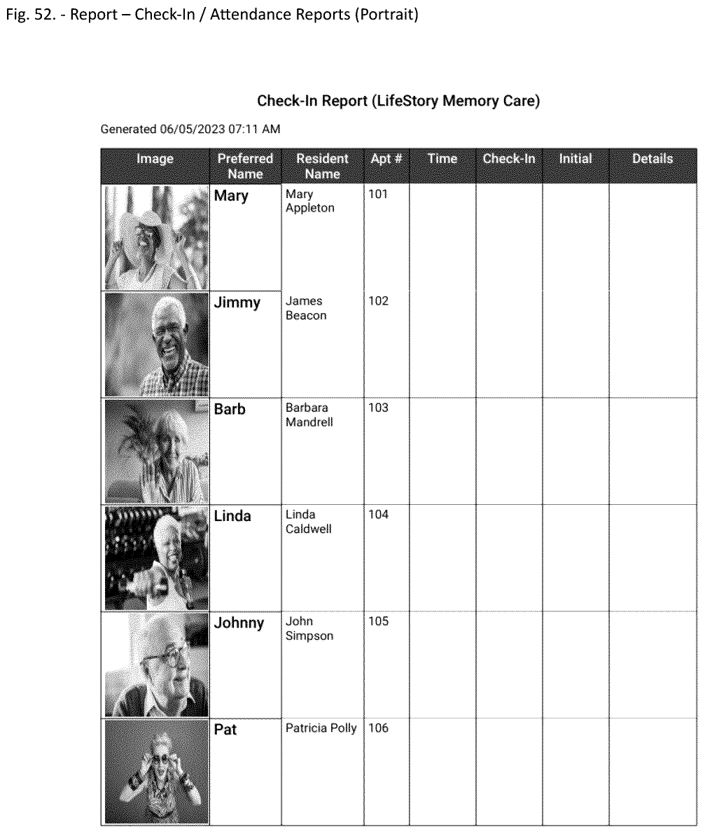

FIG. 4—Manage Access Code: This is a snapshot of the Manage Access Code dashboard that a facility/community would have access to and utilize to manage the life story and preference initial entry process within the unique new type of database structure. The dashboard displays the following information about each resident listed:
First And Last Name
Family Name
Access Code—The code that the family uses to access unique new type of database structure to enter in their loved one's life story and preferences, if desired, and if the access code is “active.”
Move-In Status—This displays whether the residents are in the pre-move-in status or moved-in status.
Status—This indicates whether the access code initially granted to the family during the pre-move-in stage is active, or inactive. The system defaults to inactive when the resident transitions from pre-move-in to moved-in.
Action—This column allows the user to choose to “Edit” the access code and family information—or—“View” the information and in this section, the user can print the access code on paper to give a family while they are in the facility/community (generally when they decide they'll be moving in their loved one and are signing the lease agreement, etc.).

FIG. 5.—Family login using access code to enter in their loved ones' life story and preferences: This is a snapshot of the page that a family granted access to the unique new type of database structure would enter in an access code that the facility/community issued to them to enter in their loved one's life story and preferences (in preparation for them moving into a dementia care facility/community).

FIG. 6.—Family life story and preference entry page instructions including facility/community name, city, state, and resident name including preferred name: This is a snapshot of the entry page for families to enter their loved one's life story and preferences into the unique new type of database structure. Families enter in the information in a few different ways including by typing text, using dropdowns to choose from a selection of responses, clicking respective check boxes that apply, and by clicking on a choice of responses at the very end including “save for later”, “submit”, etc.

FIG. 7.—General Information: The general information section of the new type of database structure has a place for the resident photo and lists the residents first name, last name, along with a field for the family to enter in their preferred name (ex: Bob versus Robert). In dementia care, residents are more comfortable being referred to their preferred name in their life which can be different than their legal name.

FIG. 8.—Family section of the resident life story and preferences: This is a snapshot of the family section of the new type of database structure where families can enter in the family information about their loved one moving into dementia care. The type of information captured in this section includes fields that capture their spouse (if applicable), siblings (if applicable), moved here from ______, etc.

FIG. 9.—Me section of the resident life story and preferences: This is a snapshot of the Me section where families can enter in the family information about their loved one moving into dementia care. Information such as the residents' occupation, education, veteran status, etc. (if applicable) is entered into the unique new type of database structure.

FIG. 10.—Dining section of the resident life story and preferences: This is a snapshot of the Dining section where families can enter in the family information about their loved one moving into dementia care. Information such as the residents' food preferences at each meal, seating preference, etc. (if applicable) is entered into the unique new type of database structure.

FIG. 11.—Favorite section of the resident life story and preferences part one: This is a snapshot of the Favorites section where families can enter in the family information about their loved one moving into dementia care. Information such as the residents' favorite activities and favorite music, pets, sporting events, teams, etc. (if applicable) is entered into the unique new type of database structure.

FIG. 12.—Favorite section of the resident life story and preferences part two: This is a snapshot of the Favorites section where families can enter in the family information about their loved one moving into dementia care. Information such as the residents' favorite activities and favorite music, pets, sporting events, teams, etc. (if applicable) is entered into the unique new type of database structure.

FIG. 13.—Important section of the resident life story and preferences: This is a snapshot of the Important section where families can enter in the family information about their loved one moving into dementia care. Information such as things important to the resident now, things they like, things they don't like, etc. (if applicable) is entered into the unique new type of database structure.

FIG. 14.—My Preferences section of the resident life story and preferences: This is a snapshot of the My Preferences section where families can enter in the family information about their loved one moving into dementia care. Information such as the residents' preferences related to personal care (typical wake up time, etc., if applicable) is entered into the unique new type of database structure.

FIG. 15.—New Type Of Database Structure Dashboard: This snapshot is what the facility/community users see when the first login to the LifeStory Connection™ application and is obtained from the unique new type of database structure. The information displayed on the new type of database structure dashboard includes:
Number Of Residents In Pre-Move-In (Expected to move-in soon)
Number Of Residents Moved-In
Number Of Residents Out Of The Building (Out with family, In rehab, etc.)
Top Activities (In order of most popular and number of residents who share that favorite)
Top Music (In order of most popular and number of residents who share that favorite)
Top Pet Interests (In order of most popular and number of residents who share that favorite)
Resident Birthdays (Current month, next month)
Resident Veteran Status (Including veteran significant other)

FIG. 16.—Pre-Move-In Submission Data: This snapshot is what the facility/community users see when they choose to look at their pre-move-in population in the unique new type of database structure. Pre-move-in means that a person/potential future resident is expected to move-in soon, so the facility/community begins to collect their life story and preference information from the family. The dashboard of the Pre-Move-In Submission Data lists all of the residents currently in the “Pre-Move-In” status including their:
First and last name
Family name
Family email
Family phone
Access status—This indicates whether the access code initially granted to the family during the pre-move-in stage is active, or inactive. The system defaults to inactive when the resident transitions from pre-move-in to moved-in.
Submitted—This column indicates whether or not the family formally “submitted” the life story and preference information to the facility/community or not. If they did not submit it, it means the facility/community either changed the resident status to move-in and the application de-activated the access code before the family submitted it—or—the facility/community chose to change the access code to inactive before move-in (ex: family notified the facility/community that they finished entering all the information they could and to please submit it on their behalf).
Action—This column allows the user to choose to “Edit” the life story and preference information—or—“View” the information and in this section, the user can print the life story, preferences, and care plan strategies in eight different ways:
   All Information—The entire life story, preferences, and care plan strategy sections
   Top 15—The top 15 most commonly accessed fields of life story, preferences, and care plan strategy information can be printed.
   Family—The family section can be printed.
   Me—The me section can be printed.
   Dining—The dining section can be printed.
   Favorites—The favorites section can be printed.
   Important—The important section can be printed.
   My Pref—The My Preferences section can be printed.

FIG. 17.—Moved-In Submission Data: This snapshot is what the facility/community users see when they choose to look at their moved-in population in the unique new type of database structure. The application lists all of the residents currently in the “move-in” status including their:
First and last name
Family name
Family email
Family phone
Publish Y/N (this determines whether the resident information will be conveyed to the tablets/devices)
Resident in building Y/N (If the resident is designated to be out of the building, the row displaying the resident will appear in yellow to stand out for the unique database users and staff/care partners viewing the resident information on the tablets/devices.
Access status—This indicates whether the access code initially granted to the family during the pre-move-in stage is active, or inactive. The system defaults to inactive when the resident transitions from pre-move-in to moved-in.
Submitted—This column indicates whether or not the family formally “submitted” the life story and preference information to the facility/community or not. If they did not submit it, it means the facility/community either changed the resident status to move-in and the application de-activated the access code before the family submitted it—or—the facility/community chose to change the access code to inactive before move-in (ex: family notified the facility/community that they finished entering all the information they could and to please submit it on their behalf).
Action—This column allows the user to choose to “Edit” the life story and preference information—or—“View” the information and in this section, the user can print the life story, preferences, and care plan strategies in eight different ways:
   All Information—The entire life story, preferences, and care plan strategy sections
   Top 15—The top 15 most commonly accessed fields of life story, preferences, and care plan strategy information can be printed.
   Family—The family section can be printed.
   Me—The me section can be printed.
   Dining—The dining section can be printed.
   Favorites—The favorites section can be printed.
   Important—The important section can be printed.
   My Pref—The My Preferences section can be printed.

FIG. 18.—Archived Submission Data: This snapshot is what the facility/community users see when they choose to look at their Archived resident population in the unique new type of database structure. The application lists all of the residents that were archived when they moved out of the facility/community.

FIG. 19.—View Resident Life Story: This snapshot shows what the facility/community user sees when they choose to “View” a residents' life story, preferences, and integrated care plan strategies from the unique new type of database structure. The choices shown on the view page offer the user an opportunity to print the information on paper in eight different ways:
All Information—The entire life story, preferences, and care plan strategy sections
Top 15—The top 15 most commonly accessed fields of life story, preferences, and care plan strategy information can be printed.
Family—The family section can be printed.
Me—The me section can be printed.
Dining—The dining section can be printed.
Favorites—The favorites section can be printed.
Important—The important section can be printed.
My Pref—The My Preferences section can be printed.

FIG. 20.—Tablet/Device home page: This snapshot is a screenshot of a tablet/device using the LifeStory Connection™ app (application) to display information from the unique new type of database structure. In this case, it's displaying the home page of the application (app) where a user (staff/care partner) can scroll through the persons/residents in their facility/community and choose one from their photo, preferred name, first name, last name, and/or apartment number.

FIG. 21.—Tablet/Device Top 15 screen part one: This snapshot is a screenshot of a tablet/device using the LifeStory Connection™ app (application) to display information from the unique new type of database structure. In this case, it displays the top 15 fields from a person/residents' life story, preferences, and integrated care plan strategies where a user (staff/care partner) can view in the course of their workday.

FIG. 22.—Tablet/Device Top 15 screen part two: This snapshot is a screenshot of a tablet/device using the LifeStory Connection™ app (application) to display information from the unique new type of database structure. In this case, it displays the top 15 fields from a person/residents' life story, preferences, and integrated care plan strategies where a user (staff/care partner) can view in the course of their workday.

FIG. 23.—Tablet/Device Family screen part one: This snapshot is a screenshot of a tablet/device using the LifeStory Connection™ app (application) to display information from the unique new type of database structure. In this case, it displays the Family fields from a person/residents' life story, preferences, and integrated care plan strategies where a user (staff/care partner) can view in the course of their workday.

FIG. 24.—Tablet/Device Family screen part two: This snapshot is a screenshot of a tablet/device using the LifeStory Connection™ app (application) to display information from the unique new type of database structure. In this case, it displays the Family fields from a person/residents' life story, preferences, and integrated care plan strategies where a user (staff/care partner) can view in the course of their workday.

FIG. 25.—Tablet/Device Me screen part one: This snapshot is a screenshot of a tablet/device using the LifeStory Connection™ app (application) to display information from the unique new type of database structure. In this case, it displays the Me fields from a person/residents' life story, preferences, and integrated care plan strategies where a user (staff/care partner) can view in the course of their workday.

FIG. 26.—Tablet/Device Me screen part two: This snapshot is a screenshot of a tablet/device using the LifeStory Connection™ app (application) to display information from the unique new type of database structure. In this case, it displays the Me fields from a person/residents' life story, preferences, and integrated care plan strategies where a user (staff/care partner) can view in the course of their workday.

FIG. 27.—Tablet/Device Dining screen part one: This snapshot is a screenshot of a tablet/device using the LifeStory Connection™ app (application) to display information from the unique new type of database structure. In this case, it displays the Dining fields from a person/residents' life story, preferences, and integrated care plan strategies where a user (staff/care partner) can view in the course of their workday.

FIG. 28.—Tablet/Device Dining screen part two: This snapshot is a screenshot of a tablet/device using the LifeStory Connection™ app (application) to display information from the unique new type of database structure. In this case, it displays the Dining fields from a person/residents' life story, preferences, and integrated care plan strategies where a user (staff/care partner) can view in the course of their workday.

FIG. 29.—Tablet/Device Favorites screen part one: This snapshot is a screenshot of a tablet/device using the LifeStory Connection™ app (application) to display information from the unique new type of database structure. In this case, it displays the Favorites fields from a person/residents' life story, preferences, and integrated care plan strategies where a user (staff/care partner) can view in the course of their workday.

FIG. 30.—Tablet/Device Favorites screen part two: This snapshot is a screenshot of a tablet/device using the LifeStory Connection™ app (application) to display information from the unique new type of database structure. In this case, it displays the Favorites fields from a person/residents' life story, preferences, and integrated care plan strategies where a user (staff/care partner) can view in the course of their workday.

FIG. 31.—Tablet/Device Important screen part one: This snapshot is a screenshot of a tablet/device using the LifeStory Connection™ app (application) to display information from the unique new type of database structure. In this case, it displays the Important fields from a person/residents' life story, preferences, and integrated care plan strategies where a user (staff/care partner) can view in the course of their workday.

FIG. 32.—Tablet/Device Important screen part two: This snapshot is a screenshot of a tablet/device using the LifeStory Connection™ app (application) to display information from the unique new type of database structure. In this case, it displays the Important fields from a person/residents' life story, preferences, and integrated care plan strategies where a user (staff/care partner) can view in the course of their workday.

FIG. 33.—Tablet/Device My Preference screen part one: This snapshot is a screenshot of a tablet/device using the LifeStory Connection™ app (application) to display information from the unique new type of database structure. In this case, it displays the My Preference fields from a person/residents' life story, preferences, and integrated care plan strategies where a user (staff/care partner) can view in the course of their workday.

FIG. 34.—Tablet/Device My Preference screen part one: This snapshot is a screenshot of a tablet/device using the LifeStory Connection™ app (application) to display information from the unique new type of database structure. In this case, it displays the My Preference fields from a person/residents' life story, preferences, and integrated care plan strategies where a user (staff/care partner) can view in the course of their workday.

FIG. 35.—Tablet/Device All Info screen part one: This snapshot is a screenshot of a tablet/device using the LifeStory Connection™ app (application) to display information from the unique new type of database structure. In this case, it displays the entire life story, preferences, and integrated care plan strategies from a person/resident where a user (staff/care partner) can view in the course of their workday.

FIG. 36.—Tablet/Device All Info screen part two: This snapshot is a screenshot of a tablet/device using the LifeStory Connection™ app (application) to display information from the unique new type of database structure. In this case, it displays the entire life story, preferences, and integrated care plan strategies from a person/resident where a user (staff/care partner) can view in the course of their workday.

FIG. 37.—Report—By Resident, By Favorite Activities: Reports are accessed by a facility/community user when logged in and are listed under the reports tab and will generate specific metrics from the information within the unique new type of database structure. This particular report lists each person/resident in the facility/community along with their favorite activities. Knowing this information helps facilities/communities quickly identify a specific person/residents' favorite activities to help to engage them in activities, foster conversation, reminisce, and to develop various approach and redirection strategies that can contribute to the care and services the facility/community offers.

FIG. 38.—Report—By Resident, By Favorite Music: Reports are accessed by a facility/community user when logged in and are listed under the reports tab and will generate specific metrics from the information within the unique new type of database structure. This particular report lists each person/resident in the facility/community along with their favorite music. Knowing this information helps facilities/communities quickly identify a specific person/residents' favorite music to help to engage them in an activity, foster conversation, reminisce, and to develop various approach and redirection strategies that can contribute to the care and services the facility/community offers.

FIG. 39.—Report—By Resident, By Religion: Reports are accessed by a facility/community user when logged in and are listed under the reports tab and will generate specific metrics from the information within the unique new type of database structure. This particular report lists each person/resident in the facility/community along with their religion. Knowing this information helps facilities/communities quickly identify a specific person/residents' religion to foster conversation, reminisce, or reference it as needed. Knowing this can also help to develop various approach and redirection strategies that can contribute to the care and services the facility/community offers.

FIG. 40.—Report—By Resident, By Favorite Sporting Events: Reports are accessed by a facility/community user when logged in and are listed under the reports tab and will generate specific metrics from the information within the unique new type of database structure. This particular report lists each person/resident in the facility/community along with their favorite sporting event. Knowing this information helps facilities/communities quickly identify a specific person/residents' favorite sporting events to help encourage them to join in on activities, foster conversation, reminiscing, and to develop various approach and redirection strategies that can contribute to the care and services the facility/community offers.

FIG. 41.—Report—By Resident, By Favorite Pets: Reports are accessed by a facility/community user when logged in and are listed under the reports tab and will generate specific metrics from the information within the unique new type of database structure. This particular report lists each person/resident in the facility/community along with their favorite pets. Knowing this information helps facilities/communities quickly identify a specific person/residents' favorite pets to help to engage them in activities, foster conversation, reminiscing, and to develop various approach and redirection strategies that can contribute to the care and services the facility/community offers.

FIG. 42.—Report—By Resident, By Occupation: Reports are accessed by a facility/community user when logged in and are listed under the reports tab and will generate specific metrics from the information within the unique new type of database structure. This particular report lists each person/resident in the facility/community along with the occupation(s) they occupied in their lives. Knowing this information helps facilities/communities quickly identify a specific person/residents' occupation(s) to help to engage them in activities, foster conversation, during reminiscing, and to develop various approach and redirection strategies that can contribute to the care and services the facility/community offers.

FIG. 43.—Report—By Category, Favorite Activities: Reports are accessed by a facility/community user when logged in and are listed under the reports tab and will generate specific metrics from the information within the unique new type of database structure. This particular report lists the favorite activities of the persons/residents in the facility/community in the order of most common, along with the respective persons/residents who share that same interest in the activity. Knowing this information helps facilities/communities contribute to their program of activities, foster conversation, help during reminiscing, etc. between persons/residents who share a similar activity interest. Additionally, know this can help to develop various approach and redirection strategies that can contribute to the care and services the facility/community offers.

FIG. 44.—Report—By Category, Favorite Music: Reports are accessed by a facility/community user when logged in and are listed under the reports tab and will generate specific metrics from the information within the unique new type of database structure. This particular report lists the favorite music of the persons/residents in the facility/community in the order of most common, along with the respective persons/residents who share that music interest. Knowing this information helps facilities/communities contribute to their program of activities, foster conversation, during reminiscing, etc. between persons/residents who shared a favorite music interest. Additionally, know this can help to develop various approach and redirection strategies that can contribute to the care and services the facility/community offers.

FIG. 45.—Report—By Category, Religion: Reports are accessed by a facility/community user when logged in and are listed under the reports tab and will generate specific metrics from the information within the unique new type of database structure. This particular report lists the religion of the persons/residents in the facility/community in the order of most common, along with the respective persons/residents who share that religion. Knowing this information helps facilities/communities plan their religious services and foster conversation between persons/residents. Additionally, know this can help to develop various approach and redirection strategies that can contribute to the care and services the facility/community offers.

FIG. 46.—Report—By Category, Favorite Sporting Events: Reports are accessed by a facility/community user when logged in and are listed under the reports tab and will generate specific metrics from the information within the unique new type of database structure. This particular report lists the favorite sporting events of the persons/residents in the facility/community in the order of most common, along with the respective persons/residents who share that interest in sporting events. Knowing this information helps facilities/communities contribute to their program of activities, foster conversation, during reminiscing, etc. between persons/residents who share a similar favorite sporting event. Additionally, know this can help to develop various approach and redirection strategies that can contribute to the care and services the facility/community offers.

FIG. 47.—Report—By Category, Favorite Pets: Reports are accessed by a facility/community user when logged in and are listed under the reports tab and will generate specific metrics from the information within the unique new type of database structure. This particular report lists the favorite pets of the persons/residents in the facility/community in the order of most common, along with the respective persons/residents who worked like those pets. Knowing this information helps facilities/communities contribute to their program of activities, foster conversation, during reminiscing, etc. between persons/residents who share a similar interest in pets. Additionally, know this can help to develop various approach and redirection strategies that can contribute to the care and services the facility/community offers.

FIG. 48.—Report—By Category, Occupation: Reports are accessed by a facility/community user when logged in and are listed under the reports tab and will generate specific metrics from the information within the unique new type of database structure. This particular report lists the occupations of the persons/residents in the facility/community in the order of most common, along with the respective persons/residents who worked in that occupation at some point in their lives. Knowing this information helps facilities/communities contribute to their program of activities, foster conversation, during reminiscing, etc. between persons/residents who shared a similar occupation in their lives. Additionally, know this can help to develop various approach and redirection strategies that can contribute to the care and services the facility/community offers.

FIG. 49.—Report—Birthdays: Reports are accessed by a facility/community user when logged in and are listed under the reports tab and will generate specific metrics from the information within the unique new type of database structure. This particular report lists the birthdays of each person/resident in the simulated facility/community from January through December. Note that no birth years are captured. Knowing this information helps facilities/communities celebrate birthdays timely and contribute to their program of activities. Additionally, know this can help to develop various approach and redirection strategies that can contribute to the care and services the facility/community offers.

FIG. 50.—Report—Veteran Status: Reports are accessed by a facility/community user when logged in and are listed under the reports tab and will generate specific metrics from the information within the unique new type of database structure. This particular report lists the veteran status of each person/resident in the simulated facility/community. Knowing this information helps facilities/communities recognize their veterans and significant others throughout the year. Additionally, know this can help to develop various approach and redirection strategies that can contribute to the care and services the facility/community offers.

FIG. 51.—Report—Check-In/Attendance Reports (Landscape): Reports are accessed by a facility/community user when logged in and are listed under the reports tab and will generate specific metrics from the information within the unique new type of database structure. This particular report is shown in landscape mode and lists all of the persons/residents in the simulated facility/community with their photo, preferred name, first and last name, and apartment number. This report can be used for multiple purposes and helps staff/care partners recognize the persons/residents in case they are new and not familiar with who-is-who.

FIG. 52.—Report—Check-In/Attendance Reports (Portrait): Reports are accessed by a facility/community user when logged in and are listed under the reports tab and will generate specific metrics from the information within the unique new type of database structure. This particular report is shown in portrait mode and lists all of the persons/residents in the simulated facility/community with their photo, preferred name, first and last name, and apartment number. This report can be used for multiple purposes and helps staff/care partners recognize the persons/residents in case they are new and not familiar with who-is-who.

### DETAILED DESCRIPTION OF THE INVENTION

A new type of database structure was developed to capture, store, analyze, organize, and convey unique fields of data to staff/care partners to provide a Person-centered approach. This application eliminates the burden currently placed on families (of loved ones who they are moving into a dementia care facility/community), facilities/communities that offer dementia care and staff/care partners who provide care and services to those with dementia. Currently:
a. Facilities/communities use paper to capture life stories and preference information from families (families of loved ones who are moving into a dementia care facility/community).
b. Facilities/communities correlate paper to identify similar interests in activities, occupations, etc. to formulate a program of activities to mirror their residents' interests.
c. Facilities/communities typically use paper, verbal communication, or other means to convey care plan strategies to staff/care partners who provide care and services to those with dementia.
d. Staff/care partners use paper to reference life stories, preferences, and care plan strategies of residents they provide care and services to who have dementia.

As a result, it's often difficult to interpret handwriting, paper is commonly lost or misplaced, and the ability to provide Person-centered care in dementia care becomes more challenging.

The LifeStory Connection™ application provides an easy-to-use tool that supports a person-centered approach in dementia care and simplifies multiple processes by utilizing a new type of database structure along with technology. With this application, each person's/residents' life story, preferences, and care plan strategies are digitized. Data analytics are generated for the facility/community that enables them to facilitate their programs more efficiently and effectively. Resident information, including integrated care plan strategies are conveyed to staff/care partners on tablets/devices (or paper) in an organized fashion with tabs for quick and easy access.

The LifeStory Connection™ application and the new processes the application offers helps staff/care partners to build trust with their residents, improve engagement, yield better outcomes, and keep families connected in dementia care. Additionally, the LifeStory Connection™ application contributes to improving each residents' experience through their journey with dementia.

The foundation of the LifeStory Connection™ application is the new type of database structure and improves the following three processes in the industry:
Family Connectivity: Families of persons with dementia who are moving their loved one into a memory care environment can provide their loved one's life story and preferences online/digitally instead of having to hand write the information on paper. Care plan strategies already identified by the family, if any, are also captured.
Data Analytics: Dementia care providers can utilize this new type of database structure that drives the application to correlate several key elements of each residents' life story and preferences to create more than 25 reports that outline similarities. Having these metrics helps them in the development of their program of activities so they best mirror their residents' interests. This tool can help to reduce aggression and depression of persons/residents in dementia care and improve their quality of life. Another report example this new type of database structure offers is an attendance report that lists each residents' name along with their photo. This can be extremely helpful to new staff/care partners who are not yet familiar with the residents they are caring for.
Care Partner Technology: With this application, staff/Care Partners are able to access each residents' life story, preferences, and care plan strategies in a simple, easy format, either on paper or a device. Each life story is separated into the following six categories/tabs on a tablet/device so staff can access the specific information they need quickly and easily:
   a. Family: Preferred name (ex: Bob versus Robert), family information, moved here from ______, etc.
   b. Me: Occupation, education, veteran status, etc.
   c. Dining: Preferences at each meal, seating preference, etc.
   d. Favorites: Favorite activities, music, pets, sporting events, teams, etc.
   e. Important: Things important to me now, things I like, things I don't like, etc.
   f. My Preferences: Preferences related to personal care (typical wake up time, etc.)

Each of these six sections includes a “notes” section for the facility/community manager(s)/director(s)/leader(s) of memory care overseeing a population of persons/residents with dementia to add the respective care plan strategies to. Some care plan strategies can be utilized in many ways and are therefore integrated within each respective category.

The application also provides two additional sections as follows:
Top 15: This section lists the 15 most popular/accessed fields of a person's/residents' life story and acts as a quick go-to reference.
All Info: this section lists the entire life story and preferences of a person/resident, one section at a time (without the Top 15 section). Care plan strategies are integrated within each category.

By utilizing the LifeStory Connection application, staff/care partners are no longer burdened with having to look through paper copies of life stories, preferences, or care plan strategies to locate individual resident information. The new database structure uniquely compartmentalizes the data into categories, runs exclusive data analytics, and integrates care plan strategies so staff/care partners can quickly and easily locate the information they need.

### SEQUENCE LISTING (IF ANY)

NOT APPLICABLE

1. A digitized method to facilitate a person-centered approach in dementia care

2. A person's life story, preferences, and integrated care plan strategies are strategically arranged to generate data analytics and categorized access in dementia care.
